(19) United States
(12) Patent Application Publication
(10) Pub. No.: US 2025/0345067 A1
(43) Pub. Date: Nov. 13, 2025

(54) NEW DISPOSABLE HEMOSTATIC CLIP

(71) Applicant: Suzhou Meidong Huicheng Precision Components Co., Ltd., Suzhou, Jiangsu (CN)

(72) Inventors: HONGJUN LIU, Suzhou, Jiangsu (CN); GANG LIU, Suzhou, Jiangsu (CN); JUN ZHU, Suzhou, Jiangsu (CN); HUA ZHAO, Suzhou, Jiangsu (CN); BO TANG, Suzhou, Jiangsu (CN)

(21) Appl. No.: 17/797,374

(22) PCT Filed: Nov. 9, 2021
(86) PCT No.: PCT/CN2021/129618

§ 371 Date: Dec. 26, 2024

(30) Foreign Application Priority Data
Jul. 22, 2021 (CN) 202110832114.2

(51) Int. Cl. A61B 17/122; A61B 17/00; A61B 17/12

(52) U.S. Cl. CPC A61B 17/1227; A61B 2017/0023; A61B 2017/00867; A61B 2017/12004

(57) ABSTRACT

The invention discloses a new disposable hemostatic clip, comprising a hemostatic clip, wherein the hemostatic clip is composed of a fixing hook, an upper clamping arm, a curved root, a lower clamping arm, a limiting block and a fixing cylindrical block; the hemostatic clip body is provided with a recovery device inside. In the invention, a recovery strip of memory metal or non-metal material is inserted into the upper clamping arm, the curved root and the lower clamping arm to replace the yielding part in the curved root of the original hemostatic clip. The recovery strip of memory metal or non-metal material has a strong yield memory, which can ensure that the elastic force between the upper clamping arm and the lower clamping arm remains unchanged when the hemostatic clip body is continuously closed and opened.

### BACKGROUND OF THE INVENTION

### 1. Field of the Invention

The invention related to the technical field of hemostatic clips, in particular to a new disposable hemostatic clip.

### 2. Description of the Related Art

Hemostatic clips are generally made of plastic products, which are closing devices used to mark, stop bleeding or close tubular tissue structures in laparoscopic abdominal surgery. The closing device can be input into the abdominal cavity through a tubular delivery device as required, and the closing device will be forcibly closed and stored in the delivery device, and then released to close the tubular tissue.

However, in the prior art, since the curved root of the hemostatic clip is made of plastic, when the curved root of the hemostatic clip is continuously closed and opened, the curved root will be prone to yield fatigue. Even if there are many modified materials, it is inevitable that the plastic itself has tissue defects. In slight cases, it will make the opening of the hemostatic clip too small to fit on the tubular tissue; in severe cases, the hemostatic clip may fall off directly from the delivery device, which will cause the delivery device to fail, and the operation will be blocked, and there is a great risk.

### SUMMARY OF THE INVENTION

The invention provides a new disposable hemostatic clip, to solve the problems that in the prior art, since the curved root of the hemostatic clip is made of plastic, when the curved root of the hemostatic clip is continuously closed and opened, the curved root will be prone to yield fatigue. Even if there are many modified materials, it is inevitable that the plastic itself has tissue defects. In light cases, it will make the opening of the hemostatic clip too small to fit on the tubular tissue; in severe cases, the hemostatic clip may fall off directly from the delivery device, which will cause the delivery device to fail, and the operation will be blocked, and there is a great risk.

In order to achieve the above purpose, the invention adopts the following technical solutions:

a new disposable hemostatic clip, comprising a hemostatic clip, wherein the hemostatic clip is composed of a fixing hook, an upper clamping arm, a curved root, a lower clamping arm, a limiting block and a fixing cylindrical block; the hemostatic clip body is provided with a recovery device inside.

As an improved solution of the technical solution according to the invention: the recovery device comprises a mounting groove; the mounting groove is provided inside the hemostatic clip body; the mounting groove is located inside the upper clamping arm, the curved root and the lower clamping arm; the mounting groove is provided with a recovery strip inside.

As an improved solution of the technical solution according to the invention: the curved root is provided with an arc-shaped groove penetratingly arranged.

As an improved solution of the technical solution according to the invention: a plurality of occluding protrusions are fixedly connected between the opposite side walls of the upper clamping arm and the lower clamping arm, and the plurality of occluding protrusions are arranged in a staggered manner.

As an improved solution of the technical solution according to the invention: the hemostatic clip body is made of polytetrafluoroethylene.

As an improved solution of the technical solution according to the invention: the fixing hook, the upper clamping arm, the curved root, the lower clamping arm, the limiting block, the fixing cylindrical block, and the plurality of occluding protrusions are integrally formed.

As an improved solution of the technical solution according to the invention: the recovery strip is fixedly connected to the outer side wall of the hemostatic clip body.

As an improved solution of the technical solution according to the invention: the recovery strip is made of memory metal or non-metal material.

Compared with the prior art, the advantageous effects of the invention are:

In the invention, a recovery strip of memory metal or non-metal material is inserted into the upper clamping arm, the curved root and the lower clamping arm to replace the yielding part in the curved root of the original hemostatic clip. The recovery strip of memory metal or non-metal material has a strong yield memory, which can ensure that the elastic force between the upper clamping arm and the lower clamping arm remains unchanged when the hemostatic clip body is continuously closed and opened. The device has a reasonable design and a clever conception tool. The use of memory metal or non-metal materials to replace the yielding part of the curved root of the original hemostatic clip greatly increases the elastic recovery strength between the upper clamping arm and the lower clamping arm, avoids the structural defects of the plastic itself, and guarantees the elastic recovery effect of the hemostatic clip.

The above description is only an overview of the technical solution of the invention. In order to make the technical solutions of the invention more clearly and implement it in accordance with the content of the description, the preferred embodiments of the invention will be described in detail hereinafter with reference to the drawings. The specific implementation of the invention will be given in detail by the following embodiments and the drawings.

### BRIEF DESCRIPTION OF THE DRAWINGS

The drawings described herein are used to provide a further understanding of the invention and constitute a part of the application. The exemplary embodiments of the invention and the description thereof are used to explain the invention, and do not constitute an improper limitation of the invention. In the figures:

FIG. 1 is a schematic diagram of the three-dimensional structure of a new type of disposable hemostatic clip proposed in Embodiment 1 according to the invention;

FIG. 2 is a schematic diagram of the front three-dimensional structure of a new type of disposable hemostatic clip proposed in Embodiment 1 according to the invention;

FIG. 3 is a schematic diagram of the front sectional structure of a new type of disposable hemostatic clip proposed in Embodiment 1 according to the invention;

FIG. 4 is a schematic diagram of a partial enlarged structure of A in FIG. 3;

FIG. 5 is a schematic diagram of the front structure of a new type of disposable hemostatic clip proposed in Embodiment 2 according to the invention.

In the drawings, the list of parts represented by each number is as follows:

1 refers to the hemostatic clip body; 2 refers to the upper clamping arm; 3 refers to the occluding protrusion; 4 refers to the limiting block; 5 refers to the fixing hook; 6 refers to the fixing cylindrical block; 7 refers to the arc-shaped groove; 8 refers to the curved root; 9 refers to the lower clamping arm; 10 refers to the recovery strip; 11 refers to the mounting groove.

### DETAILED DESCRIPTION OF THE PREFERRED EMBODIMENTS

The principles and features of the invention will be described hereinafter with reference to the drawings. The embodiments cited are only used to explain the invention, and are not used to limit the scope of the invention. In the following paragraphs, the invention will be described in more detail by way of embodiments with reference to the drawings. According to the following description and claims, the advantages and features of the invention will be clearer. It should be noted that the drawings all adopt a very simplified form and all use imprecise proportions, which are only used to conveniently and clearly assist in explaining the purpose of the embodiments of the invention.

It should be noted that when a component is referred to as being “fixed to” another component, it can be directly on another component or a centered component may also exist. When a component is referred to be “connected” to another component, it can be directly connected to another component or there may be a centered component at the same time. When a component is referred to be “installed on” another component, it can be directly installed on another component or a centered component may exist at the same time. The terms “vertical”, “horizontal”, “left”, “right” and similar expressions used herein are for illustrative purposes only.

Unless otherwise defined, all technical and scientific terms used herein have the same meaning as commonly understood by those skilled in the technical field of the invention. The terms used in the description of the invention herein are only for the purpose of describing specific embodiments, and are not intended to limit the invention. The term “and/or” as used herein includes any and all combinations of one or more related listed items.

### Embodiment 1

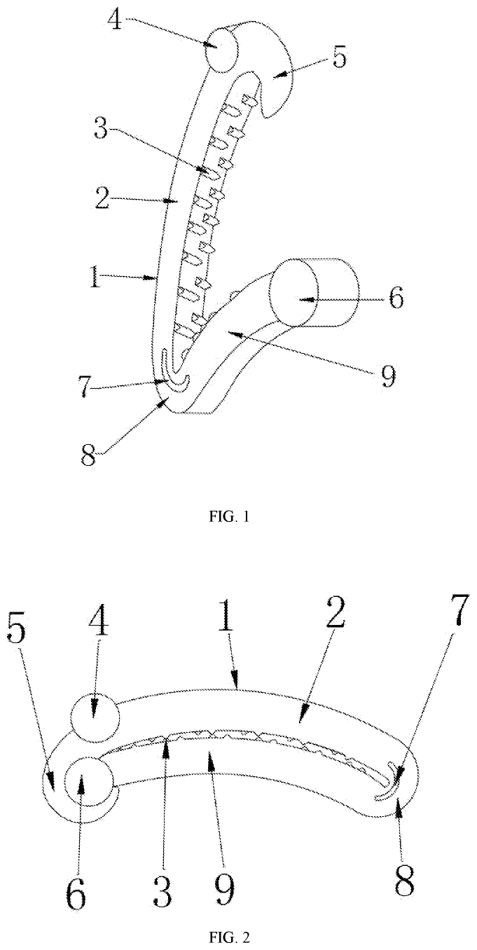

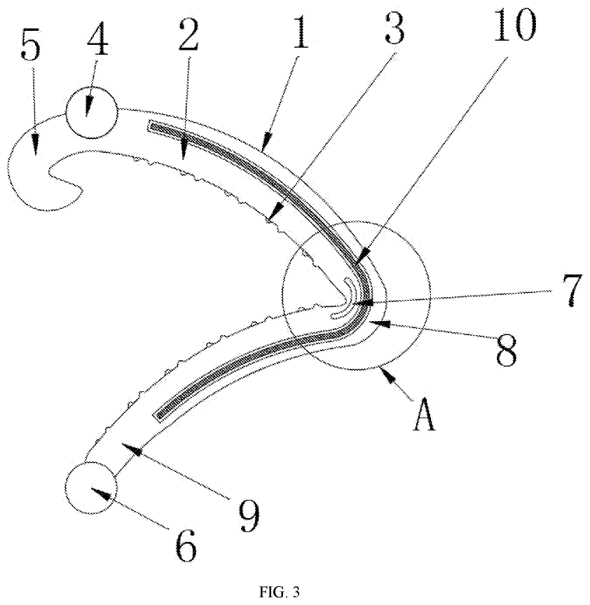

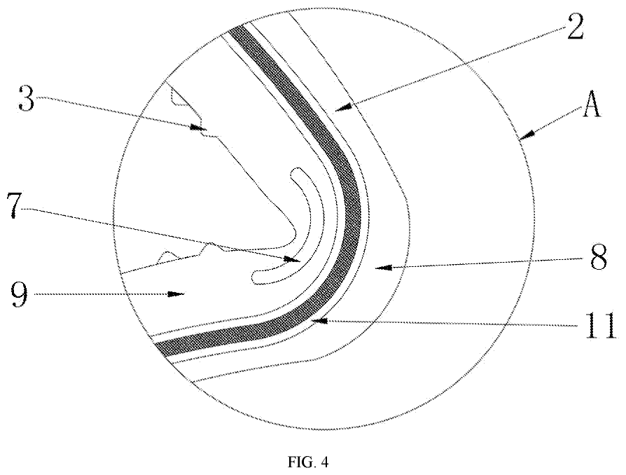

With reference to FIGS. 1-4, the embodiment of the invention provides a new disposable hemostatic clip, comprising a hemostatic clip 1, wherein the hemostatic clip 1 is composed of a fixing hook 5, an upper clamping arm 2, a curved root 8, a lower clamping arm 9, a limiting block 4 and a fixing cylindrical block 6; the hemostatic clip body 1 is provided with a recovery device inside. The recovery device comprises a mounting groove 11; the mounting groove 11 is provided inside the hemostatic clip body 1; the mounting groove 11 is located inside the upper clamping arm 2, the curved root 8 and the lower clamping arm 9; the mounting groove 11 is provided with a recovery strip 10 inside. The recovery strip 10 is made of memory metal or non-metal material. A recovery strip 10 of memory metal or non-metal material is inserted into the upper clamping arm 2, the curved root 8 and the lower clamping arm 9 to replace the yielding part in the curved root of the original hemostatic clip. The recovery strip 10 of memory metal or non-metal material has a strong yield memory, which can ensure that the elastic force between the upper clamping arm 2 and the lower clamping arm 9 remains unchanged when the hemostatic clip body 1 is continuously closed and opened.

With reference to FIG. 2, the curved root 8 is provided with an arc-shaped groove 7 penetratingly arranged. When the hemostatic clip body 1 is bent, the deformation of the curved root 8 in the hemostatic clip body 1 will cause the arc-shaped groove 7 to deform, thereby protecting the curved root 8 to be more resilient.

With reference to FIG. 1 and FIG. 2, a plurality of occluding protrusions 3 are fixedly connected between the opposite side walls of the upper clamping arm 2 and the lower clamping arm 9, and the plurality of occluding protrusions 3 are arranged in a staggered manner. When the upper clamping arm 2 and the lower clamping arm 9 are attached to each other, the plurality of occluding protrusions 3 can clamp the blood vessel clamped between the upper clamping arm 2 and the lower clamping arm 9 more firmly.

With reference to FIG. 1, the hemostatic clip body 1 is made of polytetrafluoroethylene. Polytetrafluoroethylene material has good biocompatibility and blood adaptability, no harm to human physiology, and no adverse reactions when implanted in the body.

With reference to FIG. 1, the fixing hook 5, the upper clamping arm 2, the curved root 8, the lower clamping arm 9, the limiting block 4, the fixing cylindrical block 6, and the plurality of occluding protrusions 3 are integrally formed.

The working principle of the invention is: the mounting groove 11 is provided with a recovery strip 10 inside. The recovery strip 10 is made of memory metal or non-metal material. A recovery strip 10 of memory metal or non-metal material is inserted into the upper clamping arm 2, the curved root 8 and the lower clamping arm 9 to replace the yielding part in the curved root of the original hemostatic clip. The recovery strip 10 of memory metal or non-metal material has a strong yield memory, which can ensure that the elastic force between the upper clamping arm 2 and the lower clamping arm 9 remains unchanged when the hemostatic clip body 1 is continuously closed and opened. When the blood vessel is placed between the upper clamping arm 2 and the lower clamping arm 9, the upper clamping arm 2 and the lower clamping arm 9 are attached to each other through the limiting block 4 and the fixing cylindrical block 6, as shown in FIG. 2, so that the fixing cylindrical block 6 is clamped into the fixing hook 5 to clamp the blood vessel. At the same time, the plurality of occluding protrusions 3 can clamp the blood vessel clamped between the upper clamping arm 2 and the lower clamping arm 9 more firmly.

### Embodiment 2

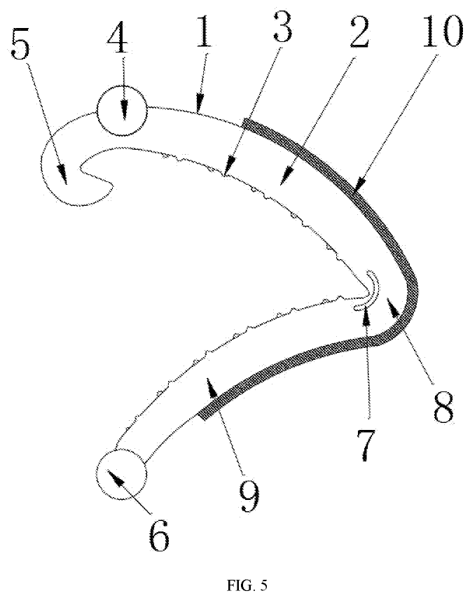

With reference to FIG. 5, the recovery strip 10 is fixedly connected to the outer side wall of the hemostatic clip body 1, for example, glued to the arc-shaped outer side wall of the hemostatic clip body 1, and the hemostatic clip body 1 is directly modified to improve the utilization of resources. The recovery strip 10 is made of memory metal or non-metal material. The memory metal or non-metal material can greatly increase the elastic recovery force between the upper clamping arm 2 and the lower clamping arm 9.

The above are only the preferred embodiments of the invention, and do not limit the invention in any form; those of ordinary skill in the art can smoothly implement the invention as shown in the drawings of the specification and described hereinabove. However, minor changes, modifications, and evolutionary equivalent changes without departing from the scope of the technical solution of the invention made by those of ordinary skill in the art are all equivalent embodiments of the invention. At the same time, any equivalent changes, modifications, and evolutions made to the above embodiments based on the essential technology of the invention shall all fall within the protection scope of the technical solution of the invention.

1. A new disposable hemostatic clip, comprising a hemostatic clip (1), wherein the hemostatic clip (1) is composed of a fixing hook (5), an upper clamping arm (2), a curved root (8), a lower clamping arm (9), a limiting block (4) and a fixing cylindrical block (6); the hemostatic clip body (1) is provided with a recovery device inside.

2. The new disposable hemostatic clip according to claim 1, wherein the recovery device comprises a mounting groove (11); the mounting groove (11) is provided inside the hemostatic clip body (1); the mounting groove (11) is located inside the upper clamping arm (2), the curved root (8) and the lower clamping arm (9); the mounting groove (11) is provided with a recovery strip (10) inside.

3. The new disposable hemostatic clip according to claim 1, wherein the curved root (8) is provided with an arc-shaped groove (7) penetratingly arranged.

4. The new disposable hemostatic clip according to claim 1, wherein a plurality of occluding protrusions (3) are fixedly connected between the opposite side walls of the upper clamping arm (2) and the lower clamping arm (9), and the plurality of occluding protrusions (3) are arranged in a staggered manner.

5. The new disposable hemostatic clip according to claim 1, wherein the hemostatic clip body (1) is made of polytetrafluoroethylene.

6. The new disposable hemostatic clip according to claim 1, wherein the fixing hook (5), the upper clamping arm (2), the curved root (8), the lower clamping arm (9), the limiting block (4), the fixing cylindrical block (6), and the plurality of occluding protrusions (3) are integrally formed.

7. The new disposable hemostatic clip according to claim 1, wherein the recovery strip (10) is fixedly connected to the outer side wall of the hemostatic clip body (1).

8. The new disposable hemostatic clip according to claim 2, wherein the recovery strip (10) is made of memory metal or non-metal material.
